(19) United States
(12) Patent Application Publication
(10) Pub. No.: US 2024/0148940 A1
(43) Pub. Date: May 9, 2024

(54) METHODS OF INHIBITION

(71) Applicants: Queensland Eye Institute Foundation, South Brisbane (AU); Universitatea de Medicinä, Farmacie, Stiinte si Tehnologie"George Emil Palade" din Târgu Mures, Târgu Mures (RO)

(72) Inventors: Leonard Azamfirei, Livezeni (RO); Traian Vasile Chirila, Robertson (AU); Shuko Suzuki, Ormeau (AU); Mark Hughes Burnard Radford, Brisbane (AU)

(21) Appl. No.: 18/504,382

(22) Filed: Nov. 8, 2023

Related U.S. Application Data
(60) Provisional application No. 63/382,791, filed on Nov. 8, 2022.

(51) Int. Cl. A61L 27/50

(52) U.S. Cl. CPC A61L 27/507; A61L 2300/428

(57) ABSTRACT

This invention relates generally to methods of inhibiting arterial rupture and the progression of an arterial aneurysm. In particular, the methods comprise applying a photosensitizer to an arterial wall and irradiating the arterial wall with photo-activating radiation to initiate crosslinking in the arterial wall and inhibit arterial rupture and the progression of an arterial aneurysm.

### RELATED APPLICATIONS

This application claims priority to U.S. Provisional Patent Application No. 63/382,791 entitled “Methods of inhibition” filed 8 Nov. 2022, the contents of which are incorporated herein by reference in their entirety.

### FIELD OF THE INVENTION

This invention relates generally to methods of inhibiting arterial rupture and the progression of an arterial aneurysm. In particular, the methods comprise applying a photosensitizer to an arterial wall and irradiating the arterial wall with photo-activating radiation to initiate crosslinking in the arterial wall and inhibit arterial rupture and the progression of an arterial aneurysm.

### BACKGROUND OF THE INVENTION

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Aortic aneurysms, and abdominal aortic aneurysms in particular, are associated with high mortality and morbidity rates, with the mortality rate being estimated as 150,000 to 200,000 deaths per year worldwide (Wang et al. (2022) Front. Cardiovasc. Med. 9: 901225). An aortic aneurysm involves the localised degenerative weakening of the aortic wall, leading to the formation of an irreversibly progressing dilatation or bulge that is at least 1.5 times greater than the diameter of the normal aorta. The spontaneous rupture of the wall can cause massive bleeding into the retroperitoneal or abdominal cavities, with fatal consequences for the subject unless an effective medical intervention is immediately available. The chance of rupture of an aortic aneurysm increases with the size of the aneurysm. Aortic aneurysms can, in some instances, develop without any symptoms and can cause sudden death due to aortic rupture with a low survival rate.

Despite a large volume of research being conducted into aneurysms, the only treatment for abdominal aortic aneurysms is repair surgery, which is either conducted to prevent rupture of the artery where the aneurysm is greater than 5 cm in females and 5.5 cm in males, or to treat a ruptured artery in an emergency setting. The surgical methods involve the insertion of prosthetic vessels through open surgery (the open surgery repair) or by stent-based procedures (endovascular aneurysm repair), which both rely on the exclusion of the aneurysmal sac from circulation (Lederle et al. (2019) N. Engl. J. Med., 380: 2126-2135). However, such surgeries are associated with severe postoperative complications, such as lung complications and heart damage, and are not suitable for subjects with small aneurysms, which are less than 5 to 5.5 cm in diameter. There are currently no treatments available for subjects with small aneurysms, with these subjects typically monitoring the size of the aneurysm using ultrasonographic imaging techniques at intervals which correspond to the size of the aneurysm being monitored (Golledge et al. (2017) Nat. Rev. Cardiol. 16: 225-242). There are no therapeutic options available which inhibit the growth of the aneurysm.

Improved therapeutic options for inhibiting the progression of aneurysms and rupture of arteries are desired.

### SUMMARY OF THE INVENTION

The present invention is predicated in part on the discovery that crosslinking of the arterial wall, particularly collagen crosslinking, can be achieved using photo-activating radiation in the presence of a photosensitizer. This crosslinking surprisingly is useful for inhibiting arterial rupture, particularly in subjects with an arterial aneurysm, such as an aortic aneurysm.

Accordingly, in one aspect, there is provided a method of inhibiting rupture of an artery in a subject, comprising applying to at least part of the arterial wall a photosensitizer that initiates crosslinking in response to photo-activating radiation, and irradiating the arterial wall with photo-activating radiation to initiate crosslinking in the arterial wall.

In some embodiments, the rupture is associated with weakening of the tunica adventitia.

In some embodiments, the artery is selected from the group consisting of the coeliac artery, superior mesenteric artery, inferior mesenteric artery, renal artery, femoral artery, tibial artery, popliteal artery, aorta, carotid artery, pulmonary artery, cerebral artery, splenic artery, hepatic artery, gastric artery, jejunal artery, ileal artery, pancreatic artery and colic artery. In particular embodiments, the artery is an aorta, such as the abdominal aorta or the thoracic aorta; especially the abdominal aorta.

In some embodiments, the photosensitizer is selected from the group consisting of riboflavin, acridine orange, Quantacure QTX, protoporphyrin IX and pharmaceutically acceptable salts, derivatives and solvates thereof. In preferred embodiments, the photosensitizer absorbs radiation at a wavelength in the range of from about 300 to about 400 nm. In particular embodiments, the photosensitizer is riboflavin or a pharmaceutically acceptable salt, derivative or solvate thereof, such as riboflavin 5′-phosphate or a pharmaceutically acceptable salt or solvate thereof.

In some embodiments, the photo-activating radiation is radiation with a wavelength in the range of from about 300 to about 400 nm, such as UV-A radiation. In particular embodiments, the photo-activating radiation is radiation with a wavelength of about 365 nm.

The method may further comprise debriding the artery to expose the arterial wall before applying the photosensitizer.

Also provided herein, in another aspect, is a method of treating or inhibiting the progression of an arterial aneurysm in a subject, comprising applying to at least part of an arterial wall comprising an aneurysm a photosensitizer that initiates crosslinking in response to photo-activating radiation, and irradiating the arterial wall with photo-activating radiation to initiate crosslinking in the arterial wall.

In some embodiments, progression of an arterial aneurysm comprises rupture of the aneurysm.

While aneurysms of all sizes are contemplated, in some embodiments, the aneurysm has a diameter of greater than about 4 cm; especially a diameter of greater than about 5 cm.

In some embodiments, the aneurysm is selected from the group consisting of an aortic aneurysm, popliteal aneurysm, splenic artery aneurysm, superior mesenteric artery aneurysm, inferior mesenteric artery aneurysm, femoral artery aneurysm, carotid aneurysm, renal artery aneurysm and hepatic artery aneurysm; especially an aortic aneurysm. In particular embodiments, the aortic aneurysm is a thoracic aortic aneurysm or abdominal aortic aneurysm; especially an abdominal aortic aneurysm.

In some embodiments, the photosensitizer is selected from the group consisting of riboflavin, acridine orange, Quantacure QTX, protoporphyrin IX and pharmaceutically acceptable salts, derivatives and solvates thereof. In specific embodiments, the photosensitizer absorbs radiation of a wavelength in the range of from about 300 to about 400 nm. In such embodiments, the photosensitizer is suitably riboflavin or a pharmaceutically acceptable salt, derivative or solvate thereof; especially riboflavin 5′-phosphate or a pharmaceutically acceptable salt or solvate thereof.

In some embodiments, the photo-activating radiation is radiation with a wavelength in the range of from about 300 to about 400 nm; especially UV-A radiation; more especially radiation with a wavelength of about 365 nm.

In some embodiments, the method may further comprise debriding the artery to expose the arterial wall before applying the photosensitizer.

### BRIEF DESCRIPTION OF THE DRAWINGS

FIG. 1 is a graph showing the stress-strain plots, covering linear, non-elastic and failure regions, recorded uniaxially for irradiated (A) and non-irradiated (B) isolated porcine abdominal aortic adventitial specimens. Sample A was irradiated with UV-A rays (365 nm wavelength), at an irradiance of 45 mW/cm² for 10 minutes in the presence of a solution of riboflavin 5′-phosphate monosodium salt in saline.

FIG. 2 is a series of graphs displaying Young's modulus and ultimate stress measured uniaxially in the linear region of non-irradiated and irradiated porcine abdominal adventitia specimens (n=30) with UV-A rays (365 nm wavelength), at an irradiance of 45 mW/cm² for 10 minutes in the presence of a solution of riboflavin 5′-phosphate monosodium salt in saline.

FIG. 3 is a series of graphs displaying Young's modulus and ultimate stress measured uniaxially in the linear region of non-irradiated and irradiated porcine abdominal adventitia specimens (n=30), which were subjected to in-vitro enzymatic degradation, either before or after irradiation with UV-A rays (365 nm wavelength). Irradiation was conducted at an irradiance of 45 mW/cm² for 10 minutes in the presence of a solution of riboflavin 5′-phosphate monosodium salt in saline.

FIG. 4 is a comparative bar graph showing the tangent modulus measured biaxially at 25% strain, in both circumferential and longitudinal directions, of non-irradiated and irradiated porcine abdominal adventitia specimens (n=23) with UV-A rays (365 nm wavelength). Irradiation was conducted at an irradiance of 45 mW/cm² for 10 minutes in the presence of a solution of riboflavin 5′-phosphate monosodium salt in saline.

FIG. 5 is a comparative bar graph displaying Cauchy stress measured biaxially at 25% strain, in both circumferential and longitudinal directions, of non-irradiated and irradiated porcine abdominal adventitia specimens (n=23) with UV-A rays (365 nm wavelength), at an irradiance of 45 mW/cm² for 10 minutes in the presence of a solution of riboflavin 5′-phosphate monosodium salt in saline.

FIG. 6 is a graph presenting the stress-strain plots measured biaxially at 25% strain, in both circumferential and longitudinal directions, prior to (non-irradiated) and after irradiation (irradiated) of a human full-thickness normal aortic wall specimen with UV-A rays (365 nm wavelength), at an irradiance of 45 mW/cm² for 10 minutes in the presence of a solution of riboflavin 5′-phosphate monosodium salt in saline.

FIG. 7 is a graph showing the stress-strain plots measured biaxially at 25% strain, in both circumferential and longitudinal directions, prior to (non-irradiated) and after irradiation (irradiated) of a human full-thickness aneurysmal aortic wall specimen with UV-A rays (365 nm wavelength). Irradiation was conducted at an irradiance of 75 mW/cm² for 10 minutes in the presence of a solution of riboflavin 5′-phosphate monosodium salt in saline.

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

By “about” is meant a quantity, level, value, number, frequency, percentage, dimension, size, amount, weight or length that varies by as much 15, 14, 13, 12, 11, 10, 9, 8, 7, 6, 5, 4, 3, 2 or 1% to a reference quantity, level, value, number, frequency, percentage, dimension, size, amount, weight or length.

The term “agent” includes a compound that induces a desired pharmacological and/or physiological effect. The term also encompasses pharmaceutically acceptable and pharmacologically active ingredients of those compounds specifically mentioned herein including but not limited to salts, esters, amides, prodrugs, active metabolites, analogues and the like. When the above term is used, then it is to be understood that this includes the active agent per se as well as pharmaceutically acceptable, pharmacologically active salts, esters, amides, prodrugs, metabolites, analogues, etc.

As used herein, the term “and/or” refers to and encompasses any and all possible combinations of one or more of the associated listed items, as well as the lack of combinations when interpreted in the alternative (or).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. Thus, the use of the term “comprising” and the like indicates that the listed integers are required or mandatory, but that other integers are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified for the listed elements. Thus, the phrase “consisting essentially of” indicates that the listed elements are required or mandatory, but that other elements are optional and may or may not be present depending upon whether or not they affect the activity or action of the listed elements.

When used herein, the term “crosslinker” or “crosslinking agent” refers to a chemical moiety that can chemically join two or more molecules, for example by covalent bonding or ionic bonding, preferably by covalent bonding. An example of a crosslinking agent is O₂ which acts as a crosslinking agent when in the form of its high energy singlet state. A crosslinker or crosslinking agent will preferably be pharmaceutically acceptable.

By “derivative” is meant a molecule, such as a small molecule, that has been derived from the basic molecule by modification, for example by conjugation or complexing with other chemical moieties as would be understood in the art. The term “derivative” also includes within its scope alterations that have been made to a parent molecule including additions or deletions that provide for functionally equivalent molecules.

By “effective amount”, in the context of inhibiting the rupture of an artery or inhibiting progression of an aortic aneurysm is meant the application of an amount of an agent or composition to an individual in need of such inhibition, that is effective for the prevention of arterial rupture or for preventing incurring a symptom (e.g. rupture) or holding in check such symptoms of aortic aneurysm. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

The phrase “inhibit the progression of” refers to a treatment which prevents the disease, disorder or condition from becoming worse, or from developing further.

By “pharmaceutically acceptable carrier” is meant a pharmaceutical vehicle comprised of a material that is not biologically or otherwise undesirable, i.e., the material may be administered to a subject along with the selected active agent without causing any or a substantial adverse reaction.

Similarly, a “pharmacologically acceptable” salt, ester, amide, prodrug, compound or derivative of a compound as provided herein is a salt, ester, amide, prodrug, compound or derivative that this not biologically or otherwise undesirable.

The term “photo-activating radiation” when used herein refers to radiation that can activate a photosensitizer to produce a chemical change in another molecule. Suitably the photosensitizer absorbs radiation at a wavelength of the photo-activating radiation. In some embodiments the radiation is radiation at a wavelength in the range of from about 300 to about 400 nm, especially UV-A radiation.

When used herein, the term “photosensitizer” refers to a molecule that, on irradiation by photo-activating radiation, produces a chemical change in another molecule through a photochemical process. Examples of “another molecule” include, for example, a crosslinker or crosslinking agent such as O₂. A photosensitizer may convert O₂ molecules from the normal O₂ triplet state to a more energetic singlet state that can initiate crosslinking, for example in tissue molecules or macromolecules. Further examples of “another molecule” include tissue molecules or macromolecules, including collagen macromolecules. A photosensitizer, after exposure to radiation and transition to a more energetic state, may also produce a chemical change in collagen and/or other tissue molecules and initiate or generate crosslinking in the tissue. The skilled person will appreciate that optimum results will be achieved when the selected photosensitizer absorbs radiation at a wavelength of the photo-activating radiation. The absorption wavelength(s) of a photosensitizer can be determined by ultraviolet/visible (UV/VIS) spectrophotometry using a commercially available UV/VIS spectrophotometer in accordance with well known procedures. A photosensitizer will preferably be pharmaceutically acceptable, non-irritant and non-toxic.

The term “pseudoaneurysm” as used herein denotes an abnormal outpouching or dilatation of arteries which are bounded only by the tunica adventitia. Pseudoaneurysms are typically caused by a rupture in the tunica intima. The injury can penetrate through the tunica media causing the blood to leak and pool as a hematoma supported by the remaining tunica adventitia.

The terms “inhibit”, “prevent”, and grammatical equivalents when used in reference to the level of a substance and/or phenomenon in a first sample relative to a second sample, mean that the quantity of substance and/or phenomenon in the first sample is lower than in the second sample by any amount that is statistically significant using any art-accepted statistical method of analysis. When these terms are used to refer to the action of a molecule or agent, the first sample may be a sample in the presence of the molecule or agent and the second sample may be a comparative sample without the molecule or agent. In one embodiment, the reduction may be determined subjectively, for example when a patient refers to their subjective perception of disease symptoms, such as pain, shortness of breath, motor symptoms, etc. In another embodiment, the reduction may be determined objectively, for example when the size of an aneurysm in a sample from a patient is smaller than in an earlier sample from the patient or from a sample from an untreated patient. In another embodiment, the quantity of substance and/or phenomenon in the first sample is at least 10% lower than the quantity of the same substance and/or phenomenon in a second sample. In another embodiment, the quantity of the substance and/or phenomenon in the first sample is at least 25% lower than the quantity of the same substance and/or phenomenon in a second sample. In yet another embodiment, the quantity of the substance and/or phenomenon in the first sample is at least 50% lower than the quantity of the same substance and/or phenomenon in a second sample. In a further embodiment, the quantity of the substance and/or phenomenon in the first sample is at least 75% lower than the quantity of the same substance and/or phenomenon in a second sample. In yet another embodiment, the quantity of the substance and/or phenomenon in the first sample is at least 90% lower than the quantity of the same substance and/or phenomenon in a second sample.

As used herein, the term “salts”, “derivative” and “solvate” include any pharmaceutically acceptable salt, derivative, or solvate or any other compound which, upon administration to the recipient, is capable of providing (directly or indirectly) a desired photosensitizer. Suitable pharmaceutically acceptable derivatives include esters, such as phosphate esters. Suitable pharmaceutically acceptable salts include salts of pharmaceutically acceptable inorganic acids such as hydrochloric, sulfuric, phosphoric, nitric, carbonic, boric, sulfamic and hydrobromic acids, or salts of pharmaceutically acceptable organic acids such as acetic, propionic, butyric, tartaric, maleic, hydroxymaleic, fumaric, citric, lactic, mucic, gluconic, benzoic, succinic, oxalic, phenylacetic, methanesulfonic, toluenesulfonic, benzenesulfonic, salicylic, sulfanilic, aspartic, glutamic, edetic, stearic, palmitic, oleic, lauric, pantothenic, tannic, ascorbic and valeric acids. Base salts include, but are not limited to, those formed with pharmaceutically acceptable cations, such as sodium, potassium, lithium, calcium, magnesium, ammonium and alkylammonium, particularly sodium. Also, basic nitrogen-containing groups may be quaternized with such agents as lower alkyl halides, such as methyl, ethyl, propyl and butyl chlorides, bromides and iodides; dialkyl sulfates such as dimethyl and diethyl sulfate; and others. Pharmacologically acceptable solvates are known in the art, and include hydrates and alcoholates. Suitably, pharmaceutically acceptable solvates include hydrates, for example monohydrates, dihydrates and trihydrates. The skilled person will understand that a photosensitizer may be in the form of a pharmaceutically acceptable salt, and/or a solvate and/or a derivative, for example riboflavin 5′-phosphate monosodium salt dihydrate and the like. The preparation of salts, derivatives and solvates can be carried out by methods well known in the art. Lists of suitable salts are found in, for example, Remington (1985) Remington's Pharmaceutical Sciences, Mack Publishing Co., Easton, Pa., 17th edition; Stahl and Wermuth (2002) Pharmaceutical Salts: Properties, Selection, and Use, Wiley-VCH; and Berge et al. (1977) Journal of Pharmaceutical Science, 66: 1-19, each of which is incorporated herein by reference in its entirety.

The term “simultaneously” denotes that the two agents are applied at substantially the same time.

The term “subject” as used herein refers to a vertebrate subject, particularly a mammalian or avian subject, for whom therapy or prophylaxis is desired. Suitable subjects include, but are not limited to, primates; avians (birds); livestock animals such as sheep, cows, horses, deer, donkeys and pigs; laboratory test animals such as rabbits, mice, rats, guinea pigs and hamsters; companion animals such as cats and dogs; and captive wild animals such as foxes, deer and dingoes. In particular embodiments, the subject is a primate, suitably a human. However, it will be understood that the aforementioned terms do not imply that symptoms are present.

As used herein, the terms “treatment”, “treating”, and the like, refer to obtaining a desired pharmacologic and/or physiologic effect. The effect may be therapeutic in terms of a partial or complete cure for a disease, disorder or condition and/or adverse effect attributable to the disease, disorder or condition. These terms also cover any treatment of a condition or disease in a subject, particularly in a human, and include: (a) inhibiting the disease or condition, i.e. arresting its development; or (b) relieving the disease or condition, i.e. causing regression of the disease or condition.

As used herein “water soluble form” refers to a chemical and/or physical form of a compound, such as a photosensitizer, where the compound or a salt, derivative, solvate and/or polymorph thereof has sufficient solubility in water at ambient temperature to achieve a concentration of from about 0.01 to about 20% w/v. Solubility can be determined using methods well known in the art.

Each embodiment described herein is to be applied mutatis mutandis to each and every embodiment unless specifically stated otherwise.

### 2. Methods of Inhibition

The inventors have determined that arterial wall tissue may be crosslinked using photo-activating radiation in the presence of a photosensitizer. Thus, the inventors have conceived that crosslinking of arterial wall tissue using photo-activating radiation in the presence of a photosensitizer is useful for inhibiting arterial rupture, particularly in subjects with an arterial aneurysm, such as an aortic aneurysm, and for treating or inhibiting the progression of an arterial aneurysm.

Accordingly, in one aspect, there is provided a method of inhibiting rupture of an artery in a subject, comprising applying to at least part of the arterial wall a photosensitizer that initiates crosslinking in response to photo-activating radiation, and irradiating the arterial wall with photo-activating radiation to initiate crosslinking in the arterial wall. Also provided is the use of a photosensitizer that initiates crosslinking in response to photo-activating radiation for inhibiting rupture of an artery in a subject, the use of a photosensitizer that initiates crosslinking in response to photo-activating radiation in the manufacture of a medicament for inhibiting rupture of an artery in a subject, and a photosensitizer that initiates crosslinking in response to photo-activating radiation for use in inhibiting rupture of an artery in a subject. Such uses may involve irradiating the arterial wall with photo-activating radiation to initiate crosslinking in the arterial wall.

In some embodiments, the photosensitizer is applied to part of the arterial wall that is susceptible to rupture. For example, the arterial wall to which the photosensitizer is applied may be weakened, thinned, bulging and/or dilated. In particular embodiments, the photosensitizer is applied to aneurysmal arterial wall tissue (i.e. the part of the arterial wall that is aneurysmal). In such embodiments, the subject may have an aneurysm in the artery.

The photosensitizer may be applied to a part of the arterial wall that is about 3 cm to about 10 cm in length (and all integer mm therebetween), especially about 3.5 cm to about 9.5 cm, about 4 cm to about 9 cm, about 4.5 cm to about 8.5 cm, about 5 cm to about 8 cm, especially about 4 cm, 4.5 cm, 5 cm, 5.5 cm, 6 cm, 6.5 cm, 7 cm, 7.5 cm, 8 cm, 8.5 cm or 9 cm. The photosensitizer is preferably applied to the entire surface of the arterial wall over this length. Accordingly, in some embodiments, the photosensitizer is suitably applied to the entire surface of the arterial wall over a length of about 3 cm to about 10 cm (and all integer mm therebetween).

In preferred embodiments, the photosensitizer is applied to the tunica adventitia (i.e. tunica externa).

In some embodiments, the rupture is associated with weakening of the tunica adventitia. That is, the tunica adventitia is weakened as compared to normal tunica adventitia tissue. In such embodiments, the photosensitizer is applied to the weakened tunica adventitia.

The photosensitizer may be applied to the arterial wall by any suitable means. Such means are well known in the art. Representative examples of which include brushing, instillation (e.g. using an applicator such as a syringe, pipette or dropper), spraying and the like, especially via brushing or spraying. In suitable embodiments, the photosensitizer is applied directly to the arterial wall, especially the tunica adventitia. In some embodiments, a solution comprising the photosensitizer is first absorbed onto an absorbent material, preferably a disposable textile pad, for example a surgical sponge, prior to application to the arterial wall. Excess photosensitizer may be removed prior to irradiation if desired using any suitable means, such as absorbent material, for example, a surgical sponge.

The method of the invention may further comprise surgical techniques to access and expose the arterial wall for photosensitizer application and irradiation. For example, in some embodiments, the method further comprises making an incision to expose the artery, debridement to expose the arterial wall prior to application of the photosensitizer and irradiation and subsequent closure of the incision after irradiation. Suitable techniques for making incisions are well known in the art and include, for example, use of a scalpel or a laser. Debriding techniques include, but are not limited to, use of a sharp instrument such as a scalpel, surgical scissors (e.g. Metzenbaum scissors), a curette and the like. Techniques and materials for effecting closure of incisions are well known in the art, and include sutures, for example silk, catgut or synthetic sutures; adhesives, for example a cyanoacrylate (e.g. 2-octyl cyanoacrylate); adhesive tapes or strips; or staples.

The method may also comprise use of a surgical drape to expose the subject arterial surface whilst protecting the remaining tissue from irradiation and photosensitizer application. In such embodiments, a hole may be cut in the surgical drape to expose the subject arterial surface whilst the remaining tissue is substantially covered by the surgical drape.

The artery may be any artery that is susceptible to rupture, such as an aneurysmal artery. In some embodiments, the artery is selected from the group consisting of the coeliac artery, superior mesenteric artery, inferior mesenteric artery, renal artery, femoral artery, tibial artery, popliteal artery, aorta, carotid artery, pulmonary artery, cerebral artery, splenic artery, hepatic artery, gastric artery, jejunal artery, ileal artery, pancreatic artery and colic artery; especially the popliteal artery, aorta, splenic artery or hepatic artery; most especially the aorta.

In particular embodiments, the artery is an aorta. While the aorta may be any segment of the aorta, including the abdominal aorta or the thoracic aorta, in particular embodiments, the aorta is the abdominal aorta.

In particular embodiments, the artery does not comprise a graft, especially a venous graft.

The photosensitizer is suitably pharmaceutically acceptable, substantially non-toxic and substantially non-irritant. In particular embodiments, the photosensitizer is water soluble. The skilled person will readily understand that different photosensitizers will absorb photosensitizing radiation of specific wavelengths according to the chemical structure of the chromophore, and will be able to match the photosensitizer to the appropriate wavelength of photosensitizing radiation. In some embodiments, the photosensitizer absorbs radiation in the ultraviolet region of the electromagnetic spectrum, especially radiation at a wavelength in the range of from about 300 to about 400 nm (and all integers therebetween). In preferred embodiments, the photosensitizer absorbs long wavelength ultraviolet radiation e.g. UV-A radiation (about 320 to about 400 nm). In particular embodiments, the photosensitizer absorbs radiation at a wavelength of about 320, 325, 330, 335, 340, 345, 350, 355, 360, 365, 370, 375, 380, 385, 390, 395 or 400 nm; especially about 365 nm. In other embodiments, the photosensitizer absorbs radiation at a wavelength of about 495 to about 570 nm (green light). In preferred embodiments, the photosensitizer has regulatory approval for food and/or drug use. While the use of more than one photosensitizer is encompassed herein, in particular embodiments, there is only a single photosensitizer molecule present.

In some embodiments, the photosensitizer comprises riboflavin or a pharmaceutically acceptable salt, derivative or solvate thereof. Riboflavin is also known as vitamin B₂, and has the IUPAC name 7,8-dimethyl-10-[(2S,3S,4R)-2,3,4,5-tetrahydroxypentyl]benzo[g]pteridine-2,4-dione. While any form of riboflavin is suitable, in preferred embodiments, riboflavin is in a water soluble form, for example as a water soluble derivative, salt or solvate, such as an alkali metal salt of riboflavin 5′-phosphate. In particular embodiments, the photosensitizer comprises a sodium salt of riboflavin 5′-phosphate or a pharmaceutically acceptable solvate thereof, such as riboflavin 5′-phosphate monosodium salt. When in the form of a solvate, the solvate is preferably a hydrate.

In some embodiments, the photosensitizer is rose bengal (4,5,6,7-tetrachloro-2′,4′,5′,7′-tetraiodofluorescein disodium salt). Preferably rose bengal is used in conjunction with irradiation by green light, i.e. a radiation wavelength of about 495 to about 570 nm.

In some embodiments, the photosensitizer is selected from the group consisting of lucigenin, acridine orange, riboflavin, Quantacure QTX (2-hydroxy-3-(3,4-dimethyl-9-oxo-9H-thioxanthen-2-yloxy)-N,N,N-trimethyl-1-propanaminium chloride), erythrosine B, protoporphyrin IX and pharmaceutically acceptable salts, derivatives and solvates thereof; especially riboflavin, acridine orange, Quantacure QTX, protoporphyrin IX or pharmaceutically acceptable salts, derivatives and solvates thereof. In such embodiments, the irradiation may have a wavelength of greater than about 300 nm, such as from about 300 nm to about 400 nm (e.g. UV-A).

In some embodiments, the photosensitizer is selected from the group consisting of Lissamine green B, Brilliant blue G, trypan blue, rose bengal and pharmaceutically acceptable salts, derivatives and solvates thereof. In such embodiments, the irradiation may have a wavelength of greater than about 400 nm, such as from about 495 nm to about 570 nm (e.g. green light).

In some embodiments, the photosensitizer is selected from the group consisting of riboflavin, rose bengal, lucigenin, acridine orange, Quantacure QTX, Lissamine green B, Brilliant blue G, trypan blue, erythrosine B, protoporphyrin IX and pharmaceutically acceptable salts, derivatives and solvates thereof; especially riboflavin, acridine orange, Quantacure QTX, protoporphyrin IX, lucigenin, rose bengal, erythrosine B and pharmaceutically acceptable salts, derivatives and solvates thereof; more especially riboflavin, acridine orange, Quantacure QTX, protoporphyrin IX and pharmaceutically acceptable salts, derivatives and solvates thereof. In preferred embodiments, the photosensitizer is riboflavin or a pharmaceutically acceptable salt, derivative or solvate thereof, such as riboflavin 5′-phosphate or a pharmaceutically acceptable salt or solvate thereof.

While the photosensitizer may be applied in a number of suitable forms, in particular embodiments, the photosensitizer is formulated in a carrier, such as a liquid or gel carrier. In such embodiments, the photosensitizer may be administered in a pharmaceutical composition comprising the photosensitizer and a pharmaceutically acceptable carrier.

The photosensitizer is preferably in a form suitable for topical application to the arterial wall, such as an emulsion, cream, solution, gel, paste or ointment; especially a solution, gel or emulsion; more especially a solution. techniques for formulation and administration may be found in, for example, Allen (Ed.) (2012) Remington: The Science and Practice of Pharmacy, The Pharmaceutical Press, London, 22^{nd} edition.

In some embodiments, the photosensitizer is formulated as an aqueous composition, for example an aqueous solution, an aqueous gel, or an oil in water emulsion, preferably as an aqueous solution. In particular embodiments, the photosensitizer is present in the composition in an amount of about 0.01 to about 20% w/v (and all integers therebetween), especially about 0.1% w/v.

In some embodiments, the carrier is an aqueous carrier. The aqueous carrier is preferably a pharmaceutically acceptable aqueous carrier. A variety of pharmaceutically acceptable aqueous carriers well known in the art may be used. Exemplary aqueous carriers include, but are not limited to, saline, water, aqueous buffer, an aqueous solution comprising water and a miscible solvent, and combinations thereof. In particular embodiments, the aqueous carrier is saline. When saline is used, it is preferably isotonic for the point of administration. For example, in some embodiments the saline comprises 0.15 to 8% w/v sodium chloride (and all one hundredth integer percentages therebetween); especially 0.18% to 7% w/v, 0.22% to 5% w/v or 0.45% to 3% w/v sodium chloride; more especially 0.5 to 2% w/v or 0.65% to 1.5% w/v sodium chloride; most especially about 0.9% w/v sodium chloride.

In some embodiments where the aqueous carrier is not isotonic, for example water, the composition may contain a tonicity agent. Any pharmaceutically acceptable tonicity agent well known in the art may be used. Suitable tonicity agents include, but are not limited to, boric acid, sodium acid phosphate buffer, sodium chloride, glucose, trehalose, potassium chloride, calcium chloride, magnesium chloride, poly(propylene glycol), glycerol, mannitol, or salts or combinations thereof. The tonicity agent may be present in the composition in an amount that provides isotonicity with the point of administration, for example in the range of from 0.02 to 15% w/v (and all one hundredth integer percentages therebetween).

In some embodiments the carrier is a buffer, wherein the buffer maintains a pH in the range of from 6 to 8, 6.5 to 7.5 or about 7. Suitable buffering agents include, but are not limited to, acetic acid, citric acid, sodium metabisulfite, histidine, sodium bicarbonate, sodium hydroxide, boric acid, borax, alkali metal phosphates, phosphate or citrate buffers, or combinations thereof. The buffering agent may be present in the composition in an amount suitable to maintain the desired pH.

The composition may further comprise a rheology modifier. The rheology modifier may be used to alter the surface tension and flow of the composition. Suitable rheology modifiers are well known in the art. For example, the rheology modifier may be selected from, but is not limited to, hyaluronic acid, chitosan, poly(vinyl alcohol), poly(ethylene glycol), poly(vinyl pyrrolidone), dextran, methylcellulose, hydroxypropylmethylcellulose, hydroxyethylcellulose, hydroxypropylcellulose, hydroxypropyl guar, acrylates such as Carbopol polymers, poloxamers, gum arabic, xanthan gum, guar gum, locust bean gum, carboxymethylcellulose, alginate, starch (from rice, corn, potato or wheat), carrageenan, konjac, aloe vera gel, agarose, pectin, tragacanth, curdlan gum, gellan gum, scleroglucan, and derivatives and combinations thereof. The rheology modifier should be present in an amount sufficient to obtain the desired viscosity of the composition. The rheology modifier may be present in an amount in the range of from about 0.5% to 5% w/v of the composition (and all one tenth integer percentages therebetween).

The composition may further comprise a surfactant. A variety of pharmaceutically acceptable surfactants well known in the art may be used. Exemplary surfactants include, but are not limited to, surfactants of the following classes: alcohols; amine oxides; block polymers; carboxylated alcohol or alkylphenol ethoxylates; carboxylic acids/fatty acids; ethoxylated arylphenols; ethoxylated fatty esters, oils, fatty amines or fatty alcohols such as cetyl alcohol; fatty esters; fatty acid methyl ester ethoxylates; glycerol esters such as glycerol monostearate; glycol esters; lanolin-based derivatives; lecithin or derivatives thereof; lignin or derivatives thereof; methyl esters; monoglycerides or derivatives thereof; poly(ethylene glycol)s; poly(propylene glycol)s; alkylphenol poly(ethylene glycol)s; alkyl mercaptan poly(ethylene glycol)s; poly(propylene glycol) ethoxylates; poly(ethylene glycol) ethers such as Cetomacrogol 1000; polymeric surfactants; propoxylated and/or ethoxylated fatty acids, alcohols or alkylphenols; protein-based surfactants; sarcosine derivatives; sorbitan derivatives such as polysorbates; sorbitol esters; esters of sorbitol polyglycol ethers; fatty acid alkylolamides; N-alkylpolyhydroxy fatty acid amide; N-alkoxypolyhydroxy fatty acid amide; alkyl polyglycosides; quaternary ammonium compounds such as benzalkonium chloride; cyclodextrins such as alpha-, beta- or gamma-cyclodextrin; sucrose or glucose esters or derivatives thereof; sulfosuccinates such as dioctyl sodium sulfosuccinate; or combinations thereof.

The composition of the invention may further comprise any other pharmaceutically acceptable excipient commonly present in pharmaceutical formulations. For example, the composition may further comprise an alcohol such as isopropanol, benzyl alcohol, cetearyl alcohol or ethanol; a polysaccharide such as chitosan, chitin, dermatan, hyaluronate, heparin, chondroitin, cyclodextrin or derivatives thereof; or combinations thereof.

Suitable water soluble photosensitizers are readily available from commercial sources, such as Sigma Aldrich, Inc. (Merck KGaA, Darmstadt, Germany) and Cayman Chemicals (Ann Arbor, Michigan, USA). For example, riboflavin 5′-phosphate sodium salt, riboflavin 5′-phosphate sodium salt hydrate and riboflavin 5′-phosphate sodium salt dihydrate are available from Sigma Aldrich and Cayman Chemicals. Photosensitizers formulated as a solution are commercially available, for example ParaCel™ (Avedro, Inc, Waltham, MA, USA) is a commercially available aqueous solution comprising 0.25% riboflavin in the form of riboflavin 5′-phosphate sodium salt, and VibeX Rapid™ (Avedro, Inc) is a commercially available solution comprising 0.1% riboflavin 5′-phosphate sodium salt.

The amount of photosensitizer applied to the arterial wall will depend on the area of arterial wall to be treated and the identity of the photosensitizer. In some embodiments, the amount of photosensitizer applied to the arterial wall is from about 1 mL to about 10 mL (and all integers therebetween) of a solution comprising from about 0.01 to about 0.5% w/v (and all integers therebetween) of photosensitizer; especially about 0.05 to about 0.2% w/v; most especially about 0.1% w/v.

The photosensitizer may be applied to the arterial wall simultaneously with or prior to irradiation, especially prior to irradiation. For example, the photosensitizer may be applied from about 30 seconds to about 45 minutes (and all seconds and minutes therebetween) prior to irradiation, such as from about 5 minutes to 40 minutes, 10 minutes to 35 minutes, 15 minutes to 30 minutes and the like, including about 10, 15, 20, 25 or 30 minutes prior to irradiation. Where required, additional photosensitizer may be applied to the arterial wall during irradiation.

Following application of the photosensitizer, the arterial wall is exposed to radiation of an appropriate wavelength for the photosensitizer used. Suitable wavelengths are discussed supra. In particular embodiments, the photosensitizer is a sodium salt of riboflavin 5′-phosphate and the radiation wavelength is in the range of from about 300 to about 400 nm (and all integers therebetween), especially UV-A radiation of a wavelength of about 320 to about 400 nm. In preferred embodiments, the radiation wavelength is about 365 nm.

A skilled person will appreciate that the irradiation time necessary to induce sufficient crosslinking in the arterial wall will be dependent on several factors including the irradiance intensity delivered by the radiation source (mW/cm²) and the beam width. In some embodiments, the irradiance is in the range of from about 10 to about 150 mW/cm² (and all integers therebetween), especially about 40 to 125, 40 to 100 or 75 to 100 mW/cm²; most especially about 75 to about 100 mW/cm².

It will also be appreciated that the radiant exposure should not be detrimental to the health of the arterial wall tissue. For example, in some embodiments, the fluence is in the range of from about 5 to about 20 J/cm² (and all integers therebetween), including about 7 to 18, 8 to 17, 9 to 16 or 10 to 15 J/cm²; especially about 10 to about 15 J/cm². The skilled person will be able to determine the duration of the exposure required based on the power of the radiation. Suitable durations may include, but are not limited to, about 10 seconds to 30 minutes (and all integer seconds and minutes therebetween), about 30 seconds to 20 minutes, 1 minute to 10 minutes or 3 minutes to 7 minutes. In some embodiments, the duration of exposure is about 1, 2, 3, 4, 5, 6, 7, 8, 9, 10, 11, 12, 13, 14 or 15 minutes.

In some embodiments, the radiation is applied using a beam profile of up to about 15 mm (including about 10, 11, 12, 13, 14 and 15 mm), for example, at a distance of from about 10 mm to about 30 mm (and all integer mm therebetween) from the surface of the arterial wall. A skilled person will appreciate that narrower radiation beams may be used if the radiation source is repositioned at time intervals to ensure that the desired part of the surface of the arterial wall is irradiated.

The delivery of the irradiation may be continuous or pulsed, especially continuous. While the entire surface of the exposed arterial wall may be irradiated, in some embodiments, the arterial wall is irradiated substantially where the photosensitizer has been applied.

The skilled person will readily appreciate that the wavelength of the photo-activating radiation will depend on the photosensitizer used. In some embodiments, the photo-activating radiation is radiation with a wavelength in the range of from about 300 to about 400 nm (and all integers therebetween), particularly UV-A radiation or radiation with a wavelength in the range of from about 320 to about 400 nm. In some embodiments, the radiation has a wavelength in the range of from about 330 to 390, 340 to 380, 350 to 370 or 360 to 370 nm. In particular embodiments, the photo-activating radiation is radiation with a wavelength of about 320, 325, 330, 335, 340, 345, 350, 355, 360, 365, 370, 375, 380, 385, 390, 395 or 400 nm; especially about 355, 360, 365, 370 or 375 nm. In particular embodiments, the photo-activating radiation is radiation with a wavelength of about 365 nm.

In further embodiments, the methods comprise irradiation in the presence of one or more crosslinking agents, preferably a pharmaceutically acceptable crosslinking agent. The skilled person will understand that a crosslinking agent will be preferably substantially non-irritant and non-toxic. A skilled person will be well aware of suitable crosslinking agents. In some embodiments, the crosslinking agent is O₂ gas. For example, in some embodiments irradiation is carried out in the presence of O₂ gas to provide O₂ concentrations greater than those present in normal atmospheric conditions or in the arterial wall.

Any radiation source that is able to deliver radiation of the selected wavelength is suitable for use in the methods of the invention. Radiation sources are known in the art, and suitable sources are commercially available. Radiation sources include those suitable for or intended for corneal crosslinking procedures such as, for example, XLink™ (Optos, Dunfermline, Scotland); CBM Vega XLink Crosslinking System (Carleton Optical, Chesham, UK); LightLink CXL™ (LightMed, San Clemente, CA, USA); UV-X™ 2000 Crosslinking System (IROC Innocross, Zurich, Switzerland); and KXL™ CrossLinking System (Avedro Waltham, MA, USA); or an Omnicure UV curing system, such as the OmniCure s1500 UV Curing System (Excelitas Technologies Corp., Waltham, MA, USA). In particular embodiments, the radiation source is an OmniCure s1500 UV Curing System. Many of these radiation sources typically have a beam width of approximately 10 to 12 mm. If irradiation of a greater area is necessary, a skilled person will be aware that repositioning of the radiation beam may be required or the radiation source instrument may be modified to produce a greater beam width, such as up to about 15 mm.

In preferred embodiments, the photosensitizer initiates crosslinking of collagen in response to photo-activating radiation.

In some embodiments, the method comprises the steps of:
a) making an incision in the subject to expose the artery;
b) debriding the outer surface of the artery;
c) applying a photosensitizer to the arterial wall;
d) applying to the exposed arterial wall a radiation beam; and
e) closing the incision.

In particular embodiments, the method comprises the steps of:
a) making an incision in the subject to expose the artery, especially the aorta;
b) debriding the outer surface of the artery;
c) applying a photosensitizer, such as an aqueous solution of riboflavin 5′-phosphate monosodium salt, to the arterial wall, and, for example, leaving the photosensitizer in contact with the arterial wall for about 30 minutes;
d) applying to the exposed arterial wall a radiation beam, preferably radiation with a wavelength in the range of from about 300 to about 400 nm, especially about 365 nm, for example, for about 10 minutes; and
e) closing the incision.

Also provided herein, in another aspect, is a method of treating or inhibiting the progression of an arterial aneurysm in a subject, comprising applying to at least part of an arterial wall comprising an aneurysm a photosensitizer that initiates crosslinking in response to photo-activating radiation, and irradiating the arterial wall with photo-activating radiation to initiate crosslinking in the arterial wall. Also provided is the use of a photosensitizer that initiates crosslinking in response to photo-activating radiation for treating or inhibiting the progression of an arterial aneurysm in a subject, the use of a photosensitizer that initiates crosslinking in response to photo-activating radiation in the manufacture of a medicament for treating or inhibiting the progression of an arterial aneurysm in a subject, and a photosensitizer that initiates crosslinking in response to photo-activating radiation for use in treating or inhibiting the progression of an arterial aneurysm in a subject. Such uses may involve irradiating the arterial wall with photo-activating radiation to initiate crosslinking in the arterial wall.

Progression of an arterial aneurysm may include, but is not limited to, an increase in the size of the aneurysm (i.e. increased arterial diameter) and/or rupture of the aneurysm. In some embodiments, progression of an arterial aneurysm comprises rupture of the aneurysm.

While the method of the invention may be used to inhibit the progression of aneurysms of all sizes, in some embodiments, the aneurysm has a diameter of greater than about 3 cm, especially greater than about 4 cm, more especially greater than about 5 cm, most especially greater than about 5.5 cm. In some embodiments, the aneurysm has a diameter in the range of from about 3 to 8 cm (and all integers therebetween), especially about 4 to 8 cm, most especially about 5 to 8 cm.

In some embodiments, the aneurysm is selected from the group consisting of an aortic aneurysm, popliteal aneurysm, splenic artery aneurysm, superior mesenteric artery aneurysm, inferior mesenteric artery aneurysm, femoral artery aneurysm, carotid aneurysm, renal artery aneurysm and hepatic artery aneurysm. In particular embodiments, the aneurysm is a popliteal aneurysm, aortic aneurysm, splenic artery aneurysm or hepatic artery aneurysm; especially an aortic aneurysm.

In particular embodiments, the aortic aneurysm is a thoracic aortic aneurysm or abdominal aortic aneurysm; especially an abdominal aortic aneurysm.

In particular embodiments, the artery comprising the aneurysm does not comprise a graft, including a venous graft.

Suitable photosensitizers and compositions thereof, means for application of the photosensitizer, amounts of the photosensitizer, irradiation conditions, radiation wavelengths, irradiation time, radiant exposure, radiation sources and additional crosslinking agents are as discussed supra.

In particular embodiments, the photosensitizer is selected from the group consisting of riboflavin, acridine orange, Quantacure QTX, protoporphyrin IX, lucigenin, rose bengal, erythrosine B and pharmaceutically acceptable salts, derivatives and solvates thereof; more especially riboflavin, acridine orange, Quantacure QTX, protoporphyrin IX and pharmaceutically acceptable salts, derivatives and solvates thereof. In specific embodiments, the photosensitizer absorbs radiation of a wavelength in the range of from about 300 to about 400 nm. In such embodiments, the photosensitizer is suitably riboflavin or a pharmaceutically acceptable salt, derivative or solvate thereof; especially riboflavin 5′-phosphate or a pharmaceutically acceptable salt or solvate thereof.

In some embodiments, the photo-activating radiation is radiation with a wavelength in the range of from about 300 to about 400 nm; especially UV-A radiation; more especially radiation with a wavelength of about 365 nm.

In some embodiments, the method further comprises debriding the artery to expose the arterial wall before applying the photosensitizer. Suitable methods for debriding the artery are discussed supra.

The photosensitizer may be applied to the aneurysmal section of the arterial wall. For example, the photosensitizer may be applied to part of the arterial wall that is about 3 cm to about 10 cm in length (and all integer mm therebetween), especially about 3.5 cm to about 9.5 cm, about 4 cm to about 9 cm, about 4.5 cm to about 8.5 cm, about 5 cm to about 8 cm, especially about 4 cm, 4.5 cm, 5 cm, 5.5. cm, 6 cm, 6.5 cm, 7 cm, 7.5 cm, 8 cm, 8.5 cm or 9 cm. The photosensitizer is preferably applied to the entire surface of the arterial wall over this length, especially a length of about 3 cm to about 10 cm.

In preferred embodiments, the photosensitizer is applied to the tunica adventitia (i.e. tunica externa).

The photosensitizer may be applied to the arterial wall simultaneously with or prior to irradiation, especially prior to irradiation. In some embodiments, the photosensitizer is applied from about 30 seconds to about 30 minutes (and all seconds and minutes therebetween) prior to irradiation, such as from about 5 minutes to 25 minutes, 10 minutes to 20 minutes, and the like, or from about 30 seconds to 5 minutes prior to irradiation. Additional photosensitizer may be applied to the arterial wall during irradiation if desired.

As discussed elsewhere herein, the method of the invention may further comprise surgical techniques to access and expose the arterial wall for photosensitizer application and irradiation. Suitable surgical techniques, such as incisions, debridement and closing incisions, are discussed supra.

In preferred embodiments, the photosensitizer initiates crosslinking of collagen in response to photo-activating radiation.

In some embodiments, the method comprises the steps of:
a) making an incision in the subject, especially in the abdomen of the subject, to expose the artery;
b) debriding the outer surface of the artery;
c) applying a photosensitizer to the arterial wall;
d) applying to the exposed arterial wall a radiation beam; and
e) closing the incision.

In particular embodiments, the method comprises the steps of:
a) making an incision in the subject, especially in the abdomen of the subject, to expose the artery, e.g. the aorta;
b) debriding the outer surface of the artery;
c) applying a photosensitizer, such as an aqueous solution of riboflavin 5′-phosphate monosodium salt, to the arterial wall and, for example, leaving the photosensitizer in contact with the arterial wall for about 30 minutes;
d) applying to the exposed arterial wall a radiation beam, preferably radiation with a wavelength in the range of from about 300 to about 400 nm, especially about 365 nm, for example, for about 10 minutes; and
e) closing the incision.

Also encompassed herein are methods for treating or inhibiting the progression of an arterial pseudoaneurysm in a subject. Such methods comprise applying to at least part of the arterial wall a photosensitizer that initiates crosslinking in response to photo-activating radiation, and irradiating the arterial wall with photo-activating radiation to initiate crosslinking in the arterial wall. Also provided is the use of a photosensitizer that initiates crosslinking in response to photo-activating radiation for treating or inhibiting the progression of an arterial pseudoaneurysm in a subject, the use of a photosensitizer that initiates crosslinking in response to photo-activating radiation in the manufacture of a medicament for treating or inhibiting the progression of an arterial pseudoaneurysm in a subject, and a photosensitizer that initiates crosslinking in response to photo-activating radiation for use in treating or inhibiting the progression of an arterial pseudoaneurysm in a subject. Such uses may involve irradiating the arterial wall with photo-activating radiation to initiate crosslinking in the arterial wall.

In particular embodiments, the tunica adventitia is intact and surrounds the hematoma.

Suitable embodiments of the method are discussed supra.

Any one of the methods and uses described herein may further comprise the crosslinking of other components within the arterial wall, such as proteoglycans (e.g. chondroitin sulfate-dermatan sulfate type and heparan sulfate type proteoglycans), either amongst themselves or between collagen and the other component (e.g. collagen and a proteoglycan). As such, the methods and uses may further comprise the application of an ancillary agent that initiates crosslinking of one or more other components within the arterial wall, especially a proteoglycan. The ancillary agent may be applied to the arterial wall simultaneously with or separately to the photosensitizer; especially simultaneously with the photosensitizer. The ancillary agent may, for example, include a photosensitizer or a functionalized proteoglycan, such as N-hydroxysuccinimide-modified chondroitin sulfate. A skilled person will readily be able to identify suitable ancillary agents.

Any one of the methods and uses described herein may involve the application of an effective amount of the photosensitizer and photo-activating radiation.

In order that the invention may be readily understood and put into practical effect, particular preferred embodiments will now be described by way of the following non-limiting examples.

### EXAMPLES

Materials and Methods

Certain experiments were performed on aortas excised from swine, based on the fact that the structure, morphology, biomechanics and function of the porcine and human cardiovascular systems are similar, as shown in Hughes (1986) Lab. Anim. Sci., 36: 348-350; Molacek et al. (2009) J. Vasc. Res., 46: 1-5; Eckhouse et al. (2013) Circulation, 128[suppl 1]: S186-S193; Lelovas et al. (2014) J. Am. Assoc. Lab. Anim. Sci., 53: 432-438; Lederman et al. (2014) Vasc. Med., 19: 167-174; Lysgaard Poulsen et al. (2016) Eur. J. Vasc. Endovasc. Surg., 52: 487-499; de Beaufort et al. (2018) Eur. J. Vasc. Endovasc. Surg., 55: 560-566; Edwards et al. (2022) Vascular, 30: 392-402.

In other experiments, human tissue aortic specimens were used, which were obtained from surgical theatre as discarded tissue.

Riboflavin and its salts, solvates and derivatives are readily available from commercial sources. For example, USP riboflavin 5′-phosphate monosodium salt is available from MilliporeSigma Co (St Louis, MO, USA) or from Cayman Chemicals (Ann Arbor, MI, USA). Collagenase is available from ThermoFisher Scientific (Rockford, IL, USA). All other chemicals, reagents or consumables are available from MilliporeSigma Co (St Louis, MO, USA).

Commercially available UV-A radiation sources include the UV Curing SystemOmniCure 1500 (ExcelitasTechnologies Corp, Waltham, MA, USA) or any of the collagen crosslinking systems commercialized by Avedro Inc (Waltham, MA, USA).

### Example 1—Crosslinking of Porcine Aortic Adventitia

Twenty-five porcine aortas from the abdominal region were used in this study, sourced from an abattoir. The aorta specimens were processed by first removing the extraneous adipose and loose connective tissue debris from the external surface using surgical scissors. One cut was then performed along the longitudinal axis of each aortic tube, in order to obtain flat rectangular sheets. A surgical scalpel was used to make an initial cornerwise-interstitial incision between adventitia and media layers, and the adventitial layer was carefully dissected and peeled off by hand. Following additional removal of medial tissue residues adhering to the internal surface, the specimens were stored for no longer than 30 minutes in saline at room temperature prior to irradiation. Rectangular strips of adventitia of about 7 cm×3 cm were soaked for 30 minutes at room temperature in a solution of riboflavin 5′-phosphate monosodium salt in saline (0.1% w/v). Each specimen was then irradiated with UV-A radiation of 365-nm wavelength produced by the UV Curing System OmniCure 1500 (Excelitas Technologies Corp., Waltham, MA, USA). The irradiance at the exposure site was monitored with the radiometer Dymax ACCU-CAL 50 (Dymax Corp., Torrington, CT, USA). The required irradiance was achieved by adjusting the distance between radiation source and target. Each side of the specimen was exposed to an irradiance of 45 mW/cm² for 10 minutes.

### Example 2—Uniaxial Tensile Measurements on the Isolated Porcine Aortic Adventitia

An Instron Materials Testing System Model #5943 (Instron, Norwood, MA, USA), equipped with a 50-N load cell, was used to measure uniaxially the tensile properties of isolated adventitia specimens (n=30), prior to and after irradiation. Irradiation was conducted in the presence of a solution of riboflavin 5′-phosphate monosodium salt in saline in accordance with the procedure of Example 1. Before measurements, each specimen was soaked in phosphate buffered saline (PBS) for at least 1 hour at room temperature. Strips (1 cm×3 cm) were cut, and their thickness and width were measured with a digital caliper (Digimax Global Inc., Toronto, Canada). Thirty measurements for specimens in each set of samples (non-irradiated and irradiated) were carried out along the longitudinal direction at a set gauge distance of 16 mm and a speed of 10 mm/min. The values for Young's moduli were computed from the recorded stress-strain plots in the linear region.

### Example 3—Stress-Strain Plots of the Isolated Porcine Aortic Adventitia

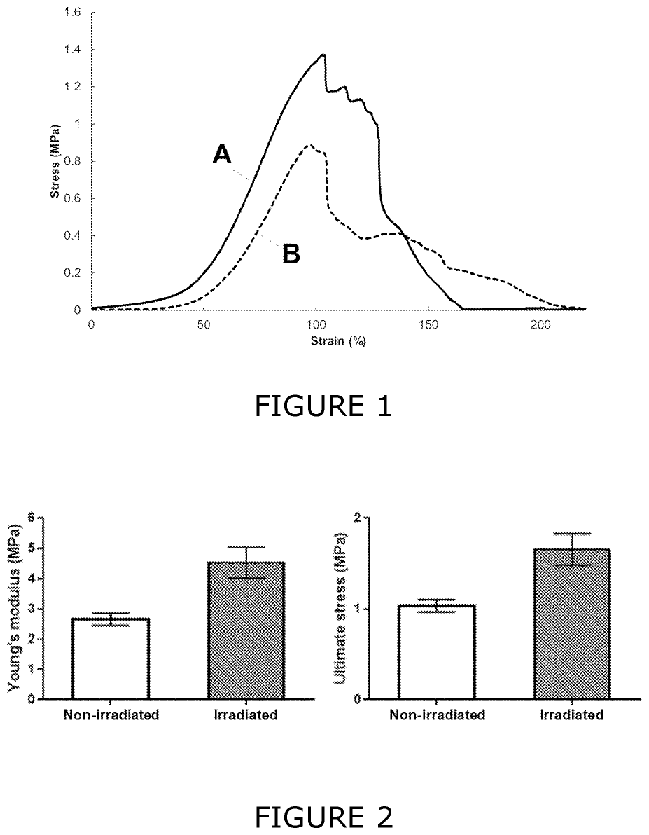

FIG. 1 shows the uniaxially recorded stress-strain plots (Example 2) of a non-irradiated porcine aortic adventitia specimen and of an irradiated specimen, demonstrating the effect of the photochemical crosslinking process on the mechanical properties of tunica adventitia. Plot “A” designates the irradiated specimen, and “B” the non-irradiated specimen, and both plots were recorded to cover the elastic, non-elastic, and failure (break) regions of the samples.

### Example 4—Uniaxial Mechanical Properties of the Isolated Porcine Aortic Adventitia

FIG. 2 shows the effect of irradiation on Young's modulus (stiffness) and ultimate stress (the maximum load at the time of rupture) of isolated porcine aortic adventitial specimens, prior to (non-irradiated) and after exposure to UV-A radiation (365 nm) at an irradiance of 45 mW/cm² for 10 minutes, on both sides of each specimen. Irradiation was conducted in the presence of a solution of riboflavin 5′-phosphate monosodium salt in saline in accordance with the procedure of Example 1. The results were expressed as mean values±standard error of the mean. The differences between non-irradiated and irradiated specimens were statistically significant at a level of less than 0.15% (p=0.0012 for modulus, p=0.0015 for stress), for n=30. For statistical comparisons, the GraphPad® Prism software (Version 6.0) was used to carry out the unpaired 2-tailed t-test.

### Example 5—In Vitro Collagenolysis of Porcine Aortic Adventitia Specimens and their Irradiation

Thirty porcine adventitial specimens were isolated and exposed to UV-A radiation as described in Example 1. Irradiation was conducted in the presence of a solution of riboflavin 5′-phosphate monosodium salt in saline in accordance with the procedure of Example 1. Non-irradiated and irradiated adventitial specimens, as sets of 10 pieces each (5 cm×3 cm), were soaked in saline for 1 hour with gentle shaking, and then placed into the wells of 6-well plates. A solution containing 20 U/mL (˜0.08 mg/mL) collagenase was prepared in Tris buffer (0.05 M, pH 7.4), which also contained calcium chloride (10 mM) and sodium azide (0.02%). To each well, 5 mL of this solution was added to cover the samples. The well plates were placed in an orbital shaker and shaken at 160 rpm for either 8 hours or 20 hours at 37° C. Three sets of 10 specimens each were processed in the following manner: (1) One set was first irradiated and then subjected to collagenolysis; (2) Another set was first subjected to collagenolysis and then irradiated; (3) The third set was subjected only to collagenolysis and was not irradiated. The remaining adventitial tissue in each well was rinsed 3 times for 10 minutes in cold water with gentle shaking, before cutting them into 1 cm×3 cm strips, in a sufficient number to provide 30 data points (n=30) for each set. Prior to mechanical testing, all samples were stored in saline.

### Example 6—Uniaxial Mechanical Properties of the Enzymatically Degraded Isolated Porcine Aortic Adventitia

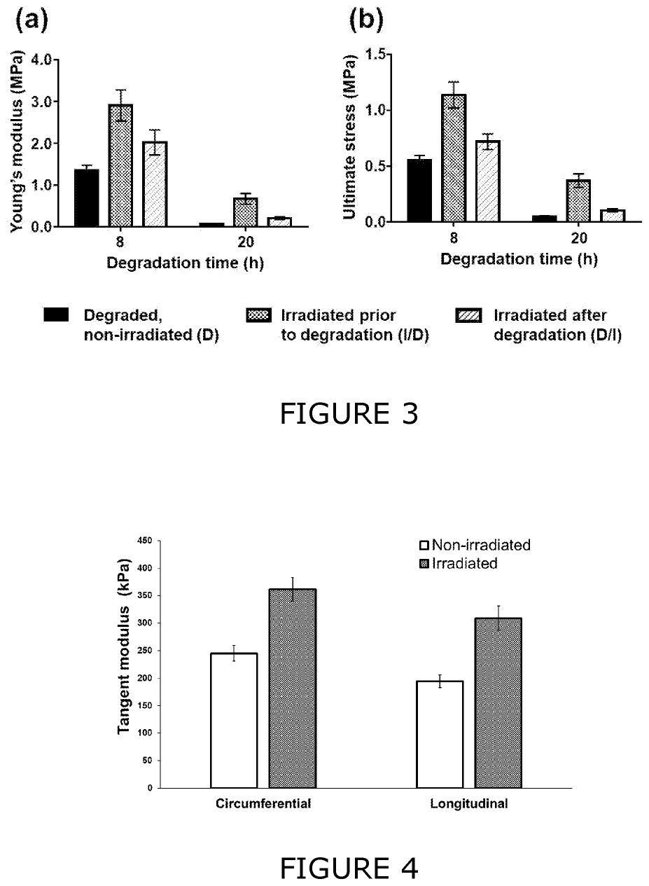

The three sets of adventitial specimens designated as (1), (2) and (3) in Example 5 were evaluated by uniaxial tensile testing following the procedure described in Example 2. FIG. 3 shows the mechanical properties of the isolated tunica adventitia specimens subjected in vitro to enzymatic degradation (collagenolysis) and selectively exposed to irradiation (UV-A, 365 nm, 45 mW/cm², 10 minutes), for n=30 in each set of samples. The bar graphs present (a) Young's modulus (stiffness) vs degradation time, and (b) ultimate stress (strength) vs degradation time. At any duration of degradation, the samples that were first irradiated and then subjected to collagenolysis (I/D) displayed the highest stiffness and strength. For the 8-hour degradation duration, the statistical difference between these samples and the degraded non-irradiated samples (D) was highly significant (p<10⁻⁴ for modulus and p<10⁻⁵ for stress), while the difference between them and the samples irradiated after degradation (D/I) showed a lower level of significance (p<0.07 for modulus and p<0.0015 for stress). For the 20-hour degradation time, the values were even more significant: p<10⁻⁶ (modulus) and p<10⁻⁷ (stress), and respectively p<10⁻⁴ (modulus) and p<10⁻⁵ (stress). The statistical differences between the degraded non-irradiated samples and those irradiated after degradation were of low significance (p˜0.2-0.5).

### Example 7—Biaxial Tensile Measurements on the Isolated Porcine Aortic Adventitia

Twenty-three porcine aortas from the abdominal region were used in this study and processed initially as described in Example 1. Irradiation was conducted in the presence of a solution of riboflavin 5′-phosphate monosodium salt in saline in accordance with the procedure of Example 1. The isolated adventitial specimens were then cut into squares of 14 mm×14 mm, and thickness was measured with a digital caliper at three spots and averaged. The tensile properties were measured and recorded biaxially in the CS BioTester™ 5000 biotesting system (CellScale Biomaterials Testing, Waterloo, ON, Canada), equipped with two 2.5-N load cells, using a set of four BioRakes™ that consisted each of 5 tungsten tines spaced evenly. All samples were submerged in a temperature-controlled bath containing PBS at 37° C., and tested by stretching the gripped area (11 mm×11 mm) to 25% of total stretch, at a rate of 1%/s. The samples were preconditioned for 10 cycles, and the tenth cycle was used for subsequent analysis. Assuming that tissue was incompressible and had negligible shear components, the Cauchy stress (σ) was determined with the equations:

σ_{θθ}=F_{θλθ}/TX_{L}

σ_{LL}=F_{LλL}/TX_{θ}

In these equations, F is the measured load, λ is the stretch, X is the initial length, T is the averaged initial thickness of the tissue sample. The subscripts θ and L designate the circumferential and longitudinal directions, respectively. The stretch was calculated as follows:

λ_{θ}=x_{θ}/X_{θ}

λ_{L}=x_{L}/X_{L}

In these equations, x represents the final length in any of the two directions.

### Example 8—Biaxial Mechanical Properties of the Isolated Porcine Aortic Adventitia

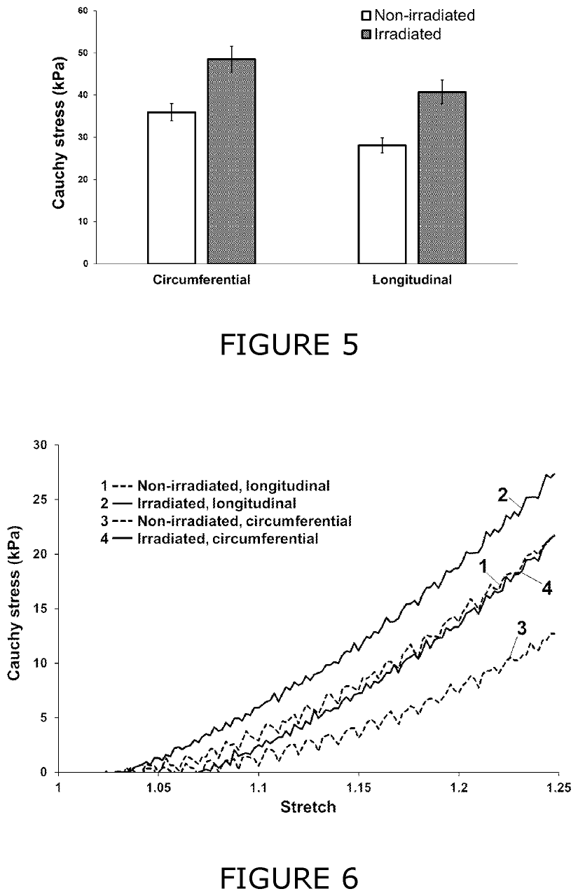

FIG. 4 shows the effect of irradiation on the tangent modulus (which is equal to Young's modulus in the linear region and represents stiffness) of isolated porcine aortic adventitial specimens, either non-irradiated or after being irradiated with UV-A rays (365 nm) at an irradiance of 45 mW/cm² for 10 minutes, on both sides of specimens (refer to Example 7). FIG. 5 shows the same effect on the stress expressed as Cauchy stress. These characteristics were evaluated for 25% stretch (strain). For statistical comparisons, the GraphPad® Prism software (Version 6.0) was used to carry out the unpaired 2-tailed t-test. The differences between non-irradiated and irradiated specimens displayed a high level of statistical significance (modulus: p=6.8×10⁻⁵ circumferentially, p=7.1×10⁻⁵ longitudinally; stress: p=1.4×10⁻³ circumferentially, p=5.3×10⁻⁴ longitudinally), for n=23.

### Example 9—Crosslinking of a Human Normal Aortic Wall Specimen

A specimen of human normal aorta was obtained as residual tissue from aortobifemoral bypass surgery. The full-thickness specimen was shaped into a square, and irradiated with UV-A (365 nm) at an irradiance of 45 mW/cm² for 10 minutes. Irradiation was conducted in the presence of a solution of riboflavin 5′-phosphate monosodium salt in saline in accordance with the procedure of Example 1. Irradiation was applied only on the adventitial side.

### Example 10—Biaxial Mechanical Properties of a Human Normal Aortic Wall Specimen

FIG. 6 shows the stress-stretch plots for a specimen of non-irradiated and irradiated full-thickness human normal (healthy) abdominal aorta (Example 9), measured biaxially in both circumferential and longitudinal directions. The measurements were performed as described in Example 7.

### Example 11—Crosslinking of a Human Aneurysmal Aortic Wall Specimen

A specimen of human aneurysmal aorta was obtained as residual tissue from the repair surgery of a mycotic abdominal aortic aneurysm. The full-thickness specimen was shaped into a square (7 mm×7 mm), and irradiated with UV-A (365 nm) at an irradiance of 75 mW/cm² for 10 minutes. Irradiation was conducted in the presence of a solution of riboflavin 5′-phosphate monosodium salt in saline in accordance with the procedure of Example 1. Irradiation was applied only on the adventitial side.

### Example 12—Biaxial Mechanical Properties of a Human Aneurysmal Aortic Wall Specimen

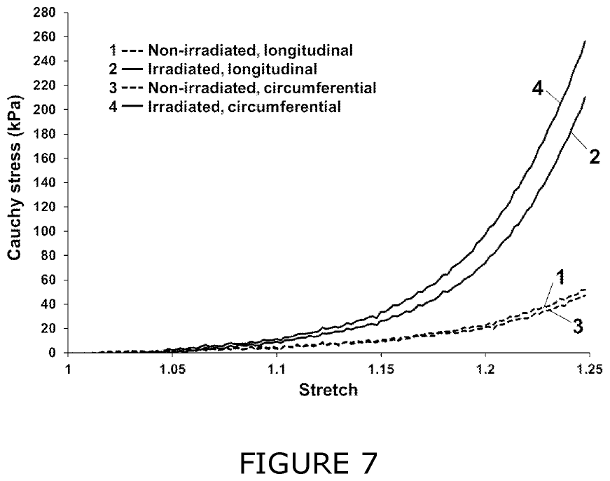

FIG. 7 shows the stress-stretch plots for a specimen of non-irradiated and irradiated full-thickness human aneurysmal abdominal aorta (Example 11), measured biaxially in both circumferential and longitudinal directions. The measurements were performed as described in Example 7.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

1. A method of inhibiting rupture of an artery in a subject, comprising applying to at least part of the arterial wall a photosensitizer that initiates crosslinking in response to photo-activating radiation, and irradiating the arterial wall with photo-activating radiation to initiate crosslinking in the arterial wall.

2. The method according to claim 1, wherein the rupture is associated with weakening of the tunica adventitia.

3. The method according to claim 1, wherein the artery is selected from the group consisting of the coeliac artery, superior mesenteric artery, inferior mesenteric artery, renal artery, femoral artery, tibial artery, popliteal artery, aorta, carotid artery, pulmonary artery, cerebral artery, splenic artery, hepatic artery, gastric artery, jejunal artery, ileal artery, pancreatic artery and colic artery.

4. The method according to claim 3, wherein the artery is an aorta.

5. The method according to claim 4, wherein the aorta is the abdominal aorta.

6. The method according to claim 1, wherein the photosensitizer is selected from the group consisting of riboflavin, acridine orange, Quantacure QTX, protoporphyrin IX and pharmaceutically acceptable salts, derivatives and solvates thereof.

7. The method according to claim 1, wherein the photosensitizer is riboflavin or a pharmaceutically acceptable salt, derivative or solvate thereof.

8. The method according to claim 1, wherein the photosensitizer is riboflavin 5′-phosphate or a pharmaceutically acceptable salt or solvate thereof.

9. The method according to claim 1, wherein the photo-activating radiation is radiation with a wavelength in the range of from about 300 to about 400 nm.

10. The method according to claim 9, wherein the photo-activating radiation is radiation with a wavelength of about 365 nm.

11. A method of treating or inhibiting the progression of an arterial aneurysm in a subject, comprising applying to at least part of an arterial wall comprising an aneurysm a photosensitizer that initiates crosslinking in response to photo-activating radiation, and irradiating the arterial wall with photo-activating radiation to initiate crosslinking in the arterial wall.

12. The method according to claim 11, wherein the aneurysm has a diameter of greater than about 4 cm.

13. The method according to claim 11, wherein the aneurysm is selected from the group consisting of an aortic aneurysm, popliteal aneurysm, splenic artery aneurysm, superior mesenteric artery aneurysm, inferior mesenteric artery aneurysm, femoral artery aneurysm, carotid aneurysm, renal artery aneurysm and hepatic artery aneurysm.

14. The method according to claim 13, wherein the aneurysm is an aortic aneurysm.

15. The method according to claim 14, wherein the aortic aneurysm is an abdominal aortic aneurysm.

16. The method according to claim 11, wherein the photosensitizer is selected from the group consisting of riboflavin, acridine orange, Quantacure QTX, protoporphyrin IX and pharmaceutically acceptable salts, derivatives and solvates thereof.

17. The method according to claim 11, wherein the photosensitizer is riboflavin or a pharmaceutically acceptable salt, derivative or solvate thereof.

18. The method according to claim 11, wherein the photosensitizer is riboflavin 5′-phosphate or a pharmaceutically acceptable salt or solvate thereof.

19. The method according to claim 11, wherein the photo-activating radiation is radiation with a wavelength in the range of from about 300 to about 400 nm.

20. The method according to claim 19, wherein the photo-activating radiation is radiation with a wavelength of about 365 nm.
